(19) United States
(12) Patent Application Publication
(10) Pub. No.: US 2024/0426786 A1
(43) Pub. Date: Dec. 26, 2024

(54) INSTANTANEOUS IN-SITU RESIN AND RESIN COMPOSITE CURE MONITORING

(71) Applicant: BAYLOR UNIVERSITY, Waco, TX (US)

(72) Inventors: David A. Jack, Waco, TX (US); Savannah Rose, Waco, TX (US); Jackson Wilkins, Waco, TX (US); Trevor Fleck, Waco, TX (US)

(73) Assignee: BAYLOR UNIVERSITY, Waco, TX (US)

(21) Appl. No.: 18/753,459

(22) Filed: Jun. 25, 2024

Related U.S. Application Data
(60) Provisional application No. 63/510,258, filed on Jun. 26, 2023.

(51) Int. Cl. G01N 29/07; G01N 29/06; G01N 33/44

(52) U.S. Cl. CPC G01N 29/07; G01N 29/0645; G01N 33/442; G01N 2291/011; G01N 2291/0235; G01N 2291/101

(57) ABSTRACT

The disclosure provides a system and method for ultrasonic non-destructive testing to monitor the curing process of a resin and filled resin materials during manufacture. The inventors have discovered that ultrasound parameters revealed in ultrasonic signal analysis reveals the degree of cure and therefore important curing stages during processing for a proper cure and allow the product to perform as intended. Generally, the ultrasound parameters include signal speed of sound and/or instantaneous peak power relative to wall-clock time of the ultrasound signal. The system and method can monitor the cure of resin and filled resin materials using instantaneous data without the need for previous data for that individual scan, can use ultrasound portably such that carbon fiber laminates can be monitored in-situ, and is conducive to isothermal cure or cure using a varying temperature profile. Thus, the invention represents a step in manufacturing of resin-curing products heretofore unavailable.

### CROSS REFERENCE TO RELATED APPLICATIONS

This application claims the benefit of U.S. Provisional Application No. 63/510,258, filed Jun. 26, 2023, entitled “Instantaneous In-situ Resin and Resin Composite Cure Monitoring”, and is incorporated herein by reference.

### STATEMENT REGARDING FEDERALLY SPONSORED RESEARCH OR DEVELOPMENT

Not applicable.

### REFERENCE TO APPENDIX

Appendices are attached and incorporated herein by reference.

### BACKGROUND OF THE INVENTION

### Field of the Invention

The disclosure generally relates to measurements of formed resin structures. More specifically, the disclosure relates to monitoring of the curing of resin systems.

### Description of the Related Art

Carbon fiber laminates, also known as carbon fiber reinforced polymers (CFRPs), are made of layers of carbon fiber fabric with resin adhering the layers together once cured. CFRPs are often used in many industrial applications due to their high strength to weight ratio. The curing process varies between thermosets used for the resin binder and a function of the curing cycle selected, and for many thermoset systems this may take several hours for the laminate to sufficiently harden such that it will not deform during separation with the tooling and may continue to cure for days to weeks after demolding. This latter time is a function of the degree of cure of the composite prior to demolding. Thus, it is useful to know how much of the laminate has cured, both to achieve the designed properties and to aid in the manufacturing process. In the present work we investigate a method using pulse-echo ultrasound to monitor the degree of cure and correlate the resulting reflected acoustic profiles to rheological testing.

Thermosets typically have three stages in curing. The first stage is when the polymer monomers grow in length, increasing the viscosity of the resin. This first stage often determines the ultimate strength of the polymer after curing. The second stage is where the reaction is dominated by cross-linking between chains. This stage often dominates the ability of the polymer to sustain damage and if taken too long, or the first stage is too short, the resulting polymer system can become brittle. The last stage is when the chemical reaction has ended, and the thermoset has completely hardened and is allowed to thermally cool. Although there is little reaction left at this stage, it is important that the system is allowed to come to thermal equilibrium slowly to prevent any embedded thermal stresses.

A known study used custom ultrasound probes to monitor resin inside the parallel plates of a rheometer that is subjected to higher temperatures. They determined the speed of sound through the resin over time has a sigmoid shape and the attenuation over time is a bell curve. They also explained how the changes in both speed of sound and attenuation relate to the three stages of the resin curing. Finally, they related the speed of sound to the storage and loss moduli of the resin, which are then related to the degree of cure from a differential scanning calorimetry (DSC) test. In another work, the same authors related the speed of sound to the loss and storage moduli in-situ within a rheometer using through-transmission ultrasound. The method presented in the study requires the instantaneous density of the resin system along with direct coupling to the resin itself with the ultrasonic transducers, by essentially fusing the transducer to that of the curing resin, making the method of limited practical use in mold fabrication.

Another known study used ultrasound to monitor the curing of a carbon fiber laminate made using the resin transfer molding (RTM) method. Like the resin study above, they found a sigmoid shaped curve for speed of sound over cure time and compared it to the degree of cure of a DSC test. They also determined that the amplitude of reflections from the laminate decreases as curing begins, and as curing is completed, the amplitude increases higher than the initial amplitude. Finally, they performed three-point bending on the laminates and found that the laminates with a higher amplitude during the cross-linking stage have better mechanical properties.

There remains a need for a system and method of in-situ monitoring of the curing of resin systems to achieve a real-time estimation of total cure time or an evaluation of complete or incomplete cure in a sample.

### BRIEF SUMMARY OF THE INVENTION

The disclosure provides a system and method for ultrasonic non-destructive testing to monitor the curing process of a resin system or a resin-filled fiber material during manufacture. The manufacture can occur in a tool, such as a surrounding mold, used to form a shape on the final manufactured resin-curing product. The system and method use ultrasound parameters that show a close relationship between the measured ultrasound acoustic parameters and the degree cure of the resin-curing product. In addition, the method can use one of several transforms, such as Fourier, Hough, or Wavelet decomposition to assist in determining the degree of cure. The method and associated system provide uniquely a direct method to monitor the cure for ideal times and stages for processing steps, thus saving costs, reducing defects, and saving time. The close correspondence of the ultrasound results to the traditional rheological profile confirms the ultrasound method usefulness. The method can use pulse echo ultrasonic technology and acoustics background knowledge to interpret data in terms of the cure process. In contrast to prior efforts, the system and method can monitor the cure of resin only or resin-filed fiber systems using instantaneous data without the need for previous data for that individual scan, can use ultrasound portably such that a resin-curing product can be monitored in-situ within the surrounding mold, and is conducive to isothermal cure or cure using a varying temperature profile.

The disclosure provides a method for ultrasonic non-destructive testing to determine a curing state of a resin-curing product, comprising: coupling an ultrasonic transducer to the resin-curing product; exciting the transducer with an ultrasound data acquisition unit coupled to the transducer to produce acoustic signals in the form of waveforms and collecting data; processing the acoustic signals into data of ultrasonic parameters; and determining the curing state of the resin-curing product from the ultrasonic parameters.

The disclosure provides a system for ultrasonic non-destructive testing to determine the curing state of a resin-curing product, comprising: a transducer configured in ultrasonic communication with the resin-curing product; an ultrasound data acquisition unit coupled to the transducer and configured to excite the transducer to generate waveforms and capture data; and an analyzer configured to analyze the waveforms into data of ultrasonic parameters indicative of a curing state of the resin-curing product.

### BRIEF DESCRIPTION OF THE SEVERAL VIEWS OF THE DRAWINGS

FIG. 1A is an illustrative side perspective view of a testing set up diagram with a mold used for performing an ultrasound acoustic process of the invention described herein for a test of resin-only curing in the mold.

FIG. 1B is an illustrative top perspective view of the testing set up diagram of FIG. 1A with layers of carbon fiber with the resin to make a cured carbon fiber laminate in the mold.

FIG. 1C is an illustrative cured product of the carbon fiber laminate of FIG. 1B removed from the mold.

FIG. 2A is a photo of illustrative ultrasound acoustic B-scan test data for the test of the resin-only cure sample relative to wall-clock time.

FIG. 2B is a photo of illustrative ultrasound acoustic B-scan test data relative to wall-clock time for the carbon fiber laminate cure sample.

FIG. 3 is a graph of illustrative ultrasound signal intensity scan data relative to signal time of a carbon fiber laminate sample.

FIG. 4A is a graph of illustrative instantaneous peak power relative to wall-clock time of the ultrasound signal reflected from the resin-only sample.

FIG. 4B is a graph of illustrative instantaneous peak power relative to wall-clock time of the ultrasound signal reflected from the carbon fiber laminate sample.

FIG. 5 is a graph of an illustrative rheological profile of parameters typically used for evaluating a curing process at room temperature relative to wall-clock time of a resin-only sample.

FIG. 6 is a graph of an illustrative comparison between the degree of cure indicated by the ultrasound data and the rheological parameter of storage modulus relative to wall-clock time.

FIG. 7 is a graph of illustrative speed of sound relative to wall-clock time of two 0.5 inch resin-only samples at 22° C.

FIG. 8 is a graph of illustrative speed of sound relative to wall-clock time of two 0.25 inch resin-only samples at 40° C.

FIG. 9 is a graph of illustrative speed of sound relative to wall-clock time of one 0.25 inch carbon fiber laminate sample at 40° C.

FIG. 10 is a graph of illustrative normalized power relative to wall-clock time of the ultrasound signal reflected from two 0.5 inch resin-only samples at 22° C.

FIG. 11 is a graph of illustrative normalized power relative to wall-clock time of the ultrasound signal reflected from one 0.25 inch carbon fiber laminate sample at 40° C.

FIG. 12 is a graph of an illustrative rheological profile of parameters relative to wall-clock time of a resin-only sample at 40° C.

FIG. 13 is a schematic diagram of an example of an ultrasonic testing system for resin curing measurements and analysis.

FIG. 14 is a schematic diagram of an example of the analyzer for the ultrasonic testing system of FIG. 13.

### DETAILED DESCRIPTION

The Figures described above, and the written description of specific aspects and functions below are not presented to limit the scope of what Applicant has invented or the scope of the appended claims. Rather, the Figures and written description are provided to teach any person skilled in the art to make and use the inventions for which patent protection is sought. Those skilled in the art will appreciate that not all features of a commercial embodiment of the inventions are described or shown for the sake of clarity and understanding. Persons of skill in this art will also appreciate that the development of an actual commercial embodiment incorporating aspects of the present disclosure will require numerous implementation-specific decisions to achieve the developer's ultimate goal for the commercial embodiment. Such implementation-specific decisions may include, and likely are not limited to, compliance with system-related, business-related, government-related, and other constraints, which may vary by specific implementation or location, or with time. While a developer's efforts might be complex and time-consuming in an absolute sense, such efforts would be, nevertheless, a routine undertaking for those of ordinary skill in this art having benefit of this disclosure. It must be understood that the inventions disclosed and taught herein are susceptible to numerous and various modifications and alternative forms. The use of a singular term, such as, but not limited to, “a,” is not intended as limiting of the number of items. Further, the various methods and embodiments of the system can be included in combination with each other to produce variations of the disclosed methods and embodiments. Discussion of singular elements can include plural elements and vice-versa. References to at least one item may include one or more items. Also, various aspects of any embodiments could be used in conjunction with each other to accomplish the understood goals of the disclosure. Unless the context requires otherwise, the term “comprise” or variations such as “comprises” or “comprising,” should be understood to imply the inclusion of at least the stated element or step or group of elements or steps or equivalents thereof, and not the exclusion of a greater numerical quantity or any other element or step or group of elements or steps or equivalents thereof. The order of steps can occur in a variety of sequences unless otherwise specifically limited. The various steps described herein can be combined with other steps, interlineated with the stated steps, and/or split into multiple steps. Some elements are nominated by a device name for simplicity and would be understood to include a system or a section, such as a controller would encompass a processor and a system of related components that are known to those with ordinary skill in the art and may not be specifically described. Various examples are provided in the description and figures that perform various functions and are non-limiting in shape, size, description, but serve as illustrative structures that can be varied as would be known to one with ordinary skill in the art given the teachings contained herein.

The disclosure provides a system and method for ultrasonic non-destructive testing to monitor the curing process of a resin and filled resin materials during manufacture. The system can include a contact transducer that can be coupled through an acoustic gel to a resin-curing product. In at least one embodiment, a pulse-echo ultrasound device, wired to the transducer, can be used for the transducer excitation and data and either controlled on the ultrasound device or from an external computer. The inventors have discovered that characterizing the ultrasonic signal quantifies the real time state of curing. The ultrasound parameters revealed in the ultrasonic signal reveal the degree of cure and therefore important curing stages during processing for a proper cure and allow the product to perform as intended. The method includes exciting the transducer coupled to a resin-curing product over a period of time and capturing the waveforms of the ultrasound signals at select moments in time, over a period of time. Generally, the waveforms are full, not gated, waveforms. The captured data is analyzed to measure differences in the signal speed of sound and/or instantaneous peak power relative to wall-clock time (that is, the time of curing) of the ultrasound signal measured with ultrasonic equipment as the resin cures for the product. In addition, an analysis of a frequency shift and/or phase shift can be used in conjunction with the signal speed of sound and/or instantaneous peak power to further characterize the signal for the state of curing. Thus, the invention represents a step in manufacturing of resin-curing products heretofore unavailable.

The analysis of the signal can also be refined by analyzing the waveforms of the reflected waves from the ultrasound signal for more subtle features such as a frequency shift or a phase shift in combination with the signal speed of sound and/or instantaneous peak power relative to wall-clock time. For example, a frequency shift of the signal at the back wall can be analyzed using a Fourier transform, such as a Short-time Fourier Transform. A phase shift of the signal at the back wall can be analyzed using a Hough transform. Each of these results can be used to help correlate the signal to the degree of cure.

The resulting captured full, not gated, waveforms can be analyzed, such as in a MATLAB script. In at least one embodiment, thermal shielding can be provided on a face of the transducer allowing for high temperature applications.

Regarding the speed of sound of the signal, the speed of sound is obtained by quantifying the difference, in time, of the reflection wave associated with the interface between the container and the resin, and the reflection wave associated with the interface between the resin and the air. This can be done by taking the onset or the time associated with the peak of each of the respective waves.

The speed of sound profile monotonically increases throughout the entire curing cycle. A simple linear model is disclosed to correlate the degree of cure to the acoustic velocity:

$$α = \frac{c - {c}_{0}}{{c}_{max} - {c}_{0}} \times 100 \qquad ( Eq . 1 )$$

where α is the degree of cure as a percentage, c is the instantaneous speed of sound, c₀ is the initial speed of sound of the resin immediately after mixing, and cₘₐₓ the speed of sound at the end of the curing.

Regarding the signal power and its intensity change during the curing process, the power within a peak over time can be calculated. The instantaneous power in the signal is a function of the instantaneous voltage as

$$\overline{P} = {\int }_{{t}_{0}}^{{t}_{1}} {f ( t )}^{2} dt \qquad ( Eq . 2 )$$

where P is power and the overbar indicates that this term for power does not include the system resistance component, thus would have units (neglecting the signal gain) of a Watt-Ohm. In Equation 2, the term f (t) is the signal voltage captured by the digitizer. Recognizing this signal has gone through a preamp, it would have units of 1/β V, where β is the gain of the signal.

The analysis of the signal can be refined with peak tracking during the curing to ensure the selected peak resulting from reflections remains the selected peak being monitored, as might occur during the process when another peak may become dominant, that is, tracking the same selected peak through the acoustic signal waveforms during the curing process independent of an intensity of the selected peak in the waveforms. Peak tracking is used to find the speed of sound over time. For each individual A scan of data, the correct peak is found and then its location and intensity are tracked over the entire test time.

The invention can yield results in an absolute basis at any time during the curing process under known conditions such as thickness of the resin-curing product through which the ultrasound wave travels and frequency of the ultrasound transducer. However, an advantageous embodiment that is practical for a manufacturing product line is to start data capture with an initial acoustic signal when starting testing to establish a baseline for the acoustic parameter(s) and measure further results of the test through the curing process to determine the ultrasonic parameters relative to the baseline acoustic parameter(s) during the curing process with a given equipment setup. With that step of characterizing the process with the relative ultrasonic parameters with that equipment, then in subsequent manufacturing of such products, the product can be analyzed at any time during the process for curing status of the product.

### Experiments

Multiple experiments were performed to support the concepts contained herein. The experiments described below show that these ultrasound parameters related to a degree of cure show a close relationship to a rheological profile of parameters indicative of the degree of cure. Thus, the invention provides a direct method to monitor the cure for ideal times and stages for processing steps.

### Materials and Method

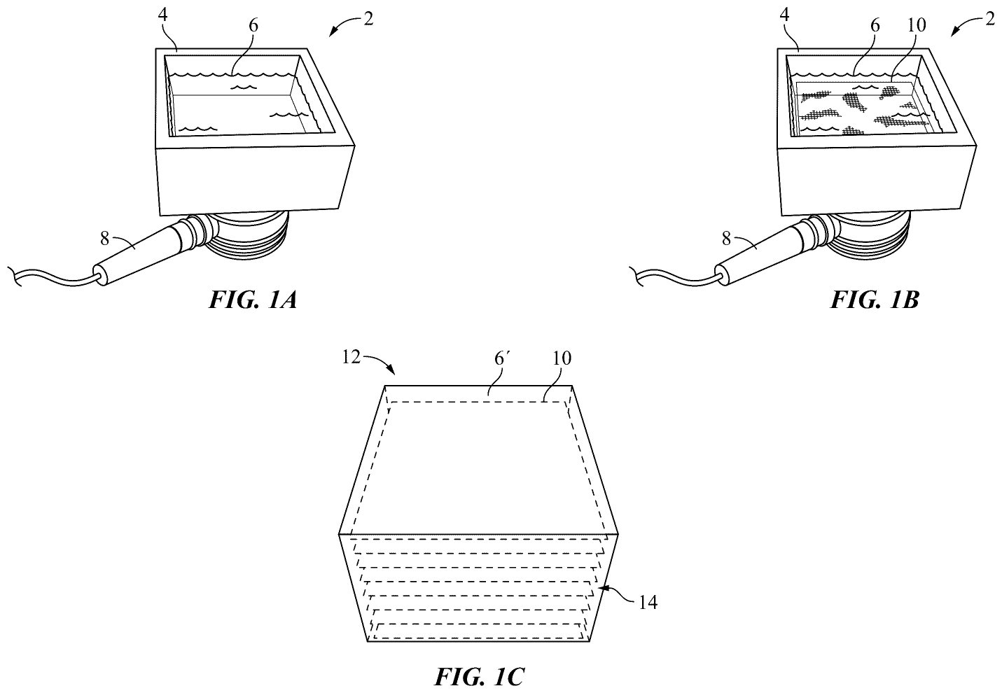

FIG. 1A is an illustrative side perspective view of a testing set up diagram with a mold used for performing an ultrasound acoustic process of the invention described herein for a test of resin-only curing in the mold. FIG. 1B is an illustrative top perspective view of the testing set up diagram of FIG. 1A with layers of carbon fiber with the resin to make a cured carbon fiber laminate in the mold. FIG. 1C is an illustrative cured product of the carbon fiber laminate of FIG. 1B removed from the mold. The ultrasonic testing setup 2 for two-part resin experiment used a 3″×3″×1″ Delrin mold 4 shown in FIG. 1A. The Delrin mold 4 was machined out to form a cavity to contain the resin 6 simulating a manufactured mold system, with the bottom surface of the Delrin being 0.25″ thick. Thus, the ultrasonic acoustic wave propagates 0.25″ through the Delrin prior to entering the resin in the mold being investigated. The side edges of the Delrin mold were machined at an angle to allow the cured resin or carbon fiber laminate to be easily removed. As observed in FIG. 1A, the Delrin mold was placed on top of a transducer 8. The transducer was an Olympus VideoScan 0.5 MHz contact transducer with a 1″ diameter and the signal coupling agent between the transducer and the bottom of the Delrin mold was a layer of EchoPure acoustic gel. The experiments used a pulse-echo method of ultrasound by an Olympus Focus PX ultrasound data acquisition unit for the transducer excitation and data capture. The resin used was a 2-part resin designed for use in the vacuum assisted resin transfer molding (VARTM) manufacturing process of Proset INF 114 resin and Proset INF 211 hardener mixed in a ratio of 3.65:1. This resin is used in the vacuum bagging industry due to its reasonable work time (˜30 minutes) and potential for both an elevated temperature curing cycle and room temperature curing cycle. In the environmental conditions for the experiment of about 22° C. for the room temperature, the resin comes to gelation after 4˜8 hours and the material supplier recommends waiting 24 hours after mixing to demold the cured product. (Gelation/gel-transition is the formation of a gel from a system with polymers. Branched polymers can form links between the chains, which lead to progressively larger polymers. At that point in the reaction, which is defined as gel point, the system loses fluidity and viscosity becomes very large.) The resin and hardener were mixed in a FlackTek mixer under a vacuum for 5 minutes to remove air from the resin. For the resin-only test, the resin was poured directly into the mold as shown.

FIG. 1B is an illustrative top perspective view of the testing set up diagram of FIG. 1A with layers of carbon fiber with the resin to make a cured carbon fiber laminate in the mold. The mold and equipment described in FIG. 1A were used for the carbon fiber laminate test. Layers of carbon fiber fabric 10 were laid in the mold between layers of the resin 6 described in FIG. 1A and then topped with a layer of resin. The testing was done in a similar fashion as in the resin-only testing in FIG. 1A.

FIG. 1C is an illustrative cured product of the carbon fiber laminate of FIG. 1B removed from the mold. The illustrative resin-curing product 12 is a cured carbon fiber laminate, formed from the carbon fiber fabric 10 and the resin 6. Other materials and layouts can be used. The product was demolded from the mold 4. Illustrative layers 14 of the carbon fiber fabric can be seen in the side of the resin-curing product.

For comparative rheological parameter testing, a second batch of resin was mixed following the identical procedure to that of the acoustic experiment and placed within a Thermo-Scientific MARS40 rheometer using a 2° cone and plate. The rheometer is set to maintain a fixed temperature of 22° C., the same as the lab conditions of the acoustic study. The temperature control prevents the exothermic reaction from self-heating the polymer. An amplitude 0.002980 rad/Nm with a frequency of 1.0 Hz was imposed on the sample, and the storage modulus, loss modulus, and the complex viscosity are tabulated as a function of time over a 24-hour test.

### Experiment 1

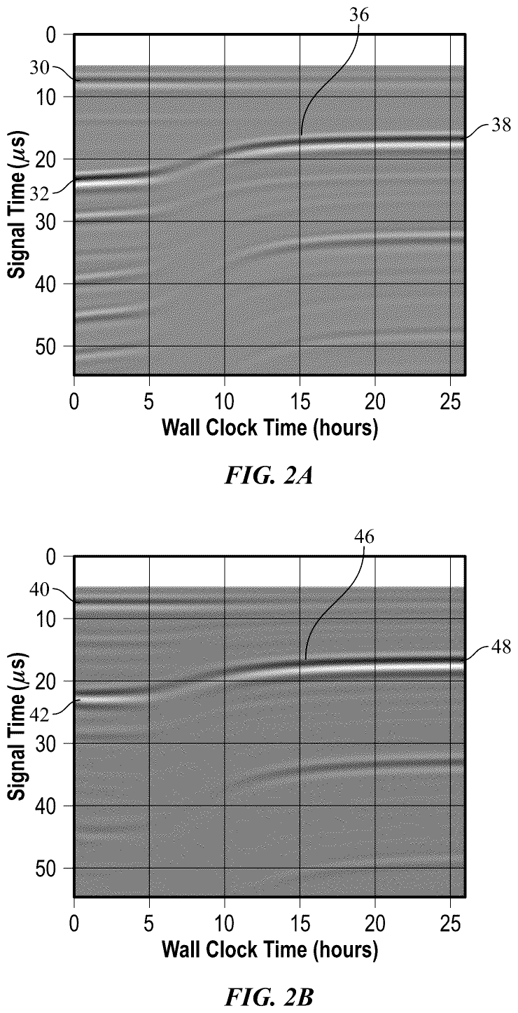

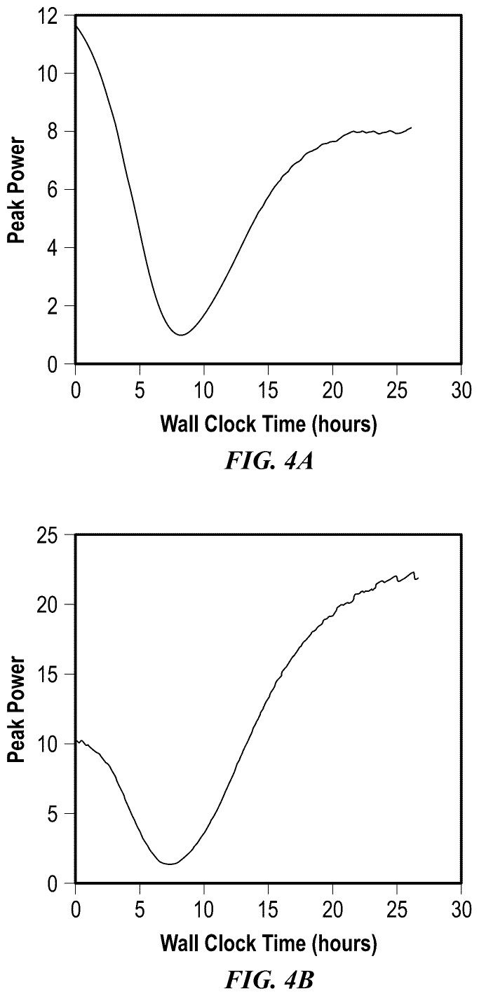

Referring to FIG. 1A, for the resin only test, the resin was poured directly into the mold as shown. To monitor the cure for both the resin test and the carbon fiber laminate test, the mold was placed on the Olympus-transducer with the acoustic gel to couple with the mold. Care was taken to level the mold relative to the contact transducer to ensure the resin would not cure at an angle. The resin curing sample was scanned for more than one full day, and the resulting captured waveforms were analyzed in a custom MATLAB script. The pulse repetition frequency for testing was 1 Hz where 4 A-scans were averaged together every second. The gain was 32 dB for the resin scan and 42 dB for the carbon fiber laminate scan to achieve an 80% signal intensity of the digitizer of the uncured system. The different gain settings were required due to more attenuation in the carbon fiber laminate, possibly due to the layered nature of the system. There also was a trigger delay of 5 us for each A-scan so as not to capture the waveform of the transducer being fired. The resin was allowed to cure according to the manufacturer's instruction while being monitored with the transducer, the signal characteristics recorded, and the results analyzed. The speed of sound of the signal in the resin-only test is shown in FIG. 2A discussed below. The signal power is shown in FIG. 4A discussed below.

### Experiment 2

Referring to FIG. 1B, to simulate a carbon fiber composite fabricated using the VARTM process, a thin layer of resin was poured into the Delrin mold and a dry woven carbon fiber ply 1.75″×1.75″ was placed in the mold as shown. Then, a thin layer or resin was applied, and another carbon fiber ply was placed in the mold. This sequence was repeated until 28 layers of carbon fiber were placed within the mold and fully wetted with resin. Finally, a thin layer of resin was poured on top of the 28th layer of carbon fiber as shown resulting in a laminate with an overall thickness of 0.51″. The same procedure and test set for Experiment 2 was used as for the resin-only test in Experiment 1, described above. The speed of sound of the signal in the resin-only test is shown in FIG. 2B discussed below. The signal power is shown in FIG. 4B discussed below.

### Results and Discussion

FIG. 2A is a photo of illustrative ultrasound acoustic B-scan test data for the test of the resin-only cure sample relative to wall-clock time. FIG. 2B is a photo of illustrative ultrasound acoustic B-scan test data relative to wall-clock time for the carbon fiber laminate cure sample. The speed of sound is obtained by looking directly at the onset of the reflection wave 30, which corresponds to the mold-to-resin interface between the Delrin mold and the resin, occurring around 8 μs at t=0 in FIG. 2A, and the next reflection wave 32 which corresponds to the resin-to-air interface, occurring at about 22 μs at t=0 in FIG. 2A for the resin-only test. Corresponding reflection waves 40 and 42, respectively, similarly appear in FIG. 2B for the carbon fiber laminate test. The B-scans shown are the collection of A-scans rotated and color coded based on amplitude where the horizontal axis is wall-clock time. The wall-clock time is in contrast to the typical horizontal axis of a position. Both sets of scan results in FIGS. 2A and 2B show an increase in the velocity of the resin as seen by the reduction in time of the signal corresponding to the resin-to-air interface. This increase is seen to be monotonic from when the resin has just been mixed at t=0 to when the resin is hardened after the 24-hour cure time has elapsed. This reflection wave 32 in FIGS. 1A and 42 in FIG. 1B occurring at the resin-to-air interface occurs around 22 μs at t=0 in FIGS. 2A and 2B), and is about 18 μs by the 15^{th} hour at point 36 through the remainder of the testing period at point 38 in FIG. 2A and slightly longer in FIG. 2B at point 46 through the remainder of the testing period at point 48. There is very little difference between the general trends in acoustic profiles of the resin sample in FIG. 2A and the carbon fiber laminate sample in FIG. 2B, because the resin changes over time but not the carbon fiber within the carbon fiber laminate.

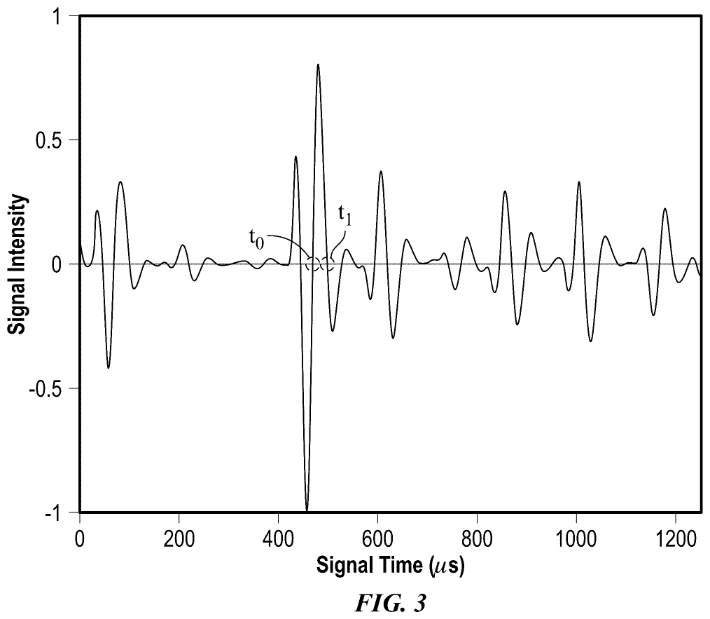

FIG. 3 is a graph of illustrative ultrasound signal intensity scan data as a waveform, relative to signal time of a carbon fiber laminate sample. FIG. 4A is a graph of illustrative instantaneous peak power relative to wall-clock time of the ultrasound signal reflected from the resin-only sample. FIG. 4B is a graph of illustrative instantaneous peak power relative to wall-clock time of the ultrasound signal reflected from the carbon fiber laminate sample. A fixed width gate, in time, during the entire test is selected in the present study that floats around the reflection wave at the resin-to-air interface, an example of which is shown in FIG. 3 at t=0 hours into the carbon fiber resin scan where t₀ and t₁ represent, respectively, the lower and upper bounds for the gate. The power is then defined as the integral of the signal over the gate, given as:

$$\overline{P} = {\int }_{{t}_{0}}^{{t}_{1}} {f ( t )}^{2} dt \qquad ( Eq . 3 )$$

In FIGS. 4A and 4B, both curves begin high when the polymer is in the liquid state. The polymer chains elongate as the system moves toward gelation as observed during the second to the fourth hours, and the peak amplitude of the acoustic signal weakens in FIGS. 2A and 3B while the speed of sound increases in FIGS. 2A and 2B. As cross-linking in gelation begins the signal power decreases until a minimum occurs around the eighth hour. As time continues past the eighth hour, the signal power increases throughout the remainder of the testing. There is an inflection point in the power curves around the fourteenth hour. The relative peaks of the power curve in the liquid state as compared to the hardened state are different for the resin-only system, shown in FIG. 4A, compared to the carbon fiber laminate sample shown in FIG. 4B. It is believed that the presence of the carbon fibers may allow for the easier transmission of acoustic signals as compared to the hardened resin.

### Experiment 3

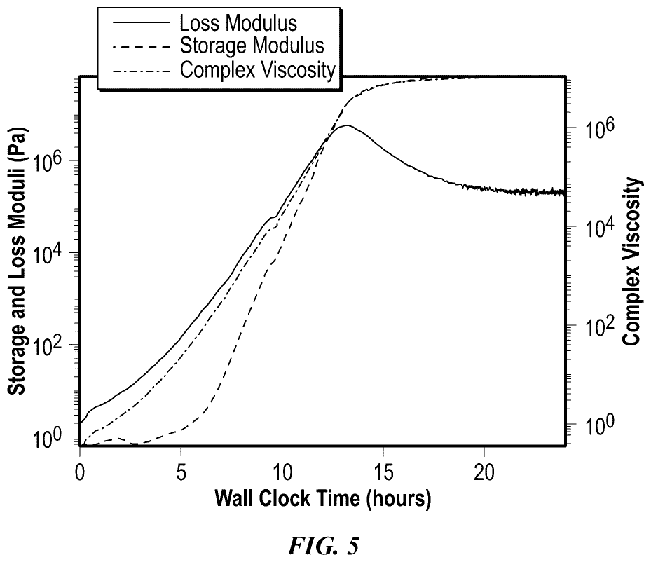

FIG. 5 is a graph of an illustrative rheological profile of parameters typically used for evaluating a curing process at room temperature relative to wall-clock time of a resin-only sample. In addition to performing the acoustic Experiments 1 and 2 above according to the invention, testing was also performed using the rheometer set up described above during curing of a separate batch of resin with the same make up as the acoustic experiments. The testing with the rheometer was according to standardized procedures for measuring rheological parameters including loss modulus, storage modulus, and complex viscosity. The standardized rheological parameters provide a comparison to the ultrasound parameters of the invention.

The polymer resin is initially in a liquid state, as indicated by the low values for each of the three material parameters. The storage modulus and complex viscosity follow similar trends and monotonically increase throughout the test. The loss modulus and complex viscosity follow very similar trends until the loss modulus peak. A peak of the loss modulus occurs around the thirteenth hour.

By comparison, an inflection point in the curves of FIGS. 3A and 3B also occurs around similar hours. Similarly, the crossover point for the storage and loss moduli in FIG. 5 occurs during the increasing slope for the power in FIGS. 3A and 3B which follows the minimum power value. Following the crossover point time, the power continues increasing, but the rate of increase gradually decreases until it plateaus. This trait corresponds to a rheological profile described below where the storage modulus also increases, but eventually plateaus at the very end of the cure.

Thus, the tests show a correlation between the acoustic intensity strength and the storage and loss moduli. Further, a transition of the resin from a rheological fluid to a viscoelastic solid, as noted by the storage modulus exceeding 1 MPa around the twelfth hour occurs around the twelfth hour of rheological testing and is close to the loss modulus achieving its peak value around the thirteenth hour. By comparison, the acoustic signal power in FIGS. 3A and 3B starts to increase around the eighth hour of testing, several hours before the cross-over point for the viscoelastic data. Thus, the acoustic signal power is believed to be a leading indicator of the loss modulus based upon this set of results.

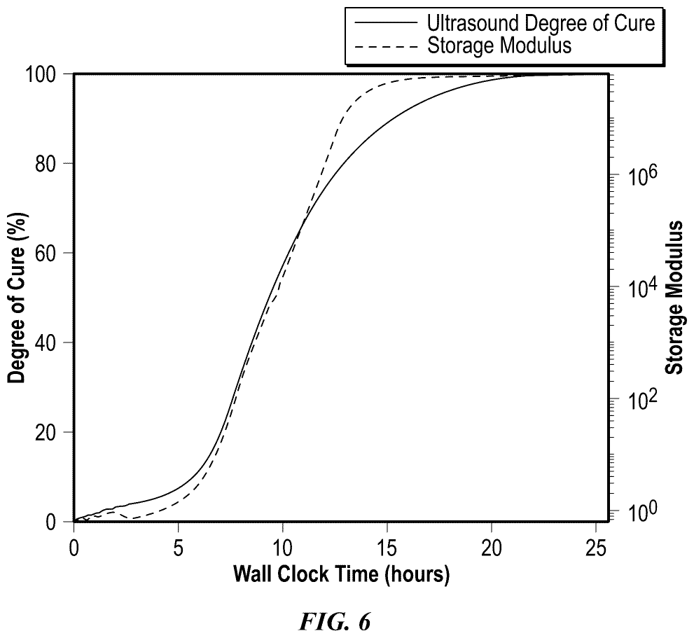

FIG. 6 is a graph of an illustrative comparison between the degree of cure indicated by the ultrasound data and the rheological parameter of storage modulus relative to wall-clock time. The degree of cure obtained using Equation 1 above during the test is plotted. The storage modulus from the rheological testing is also plotted. The ultrasound degree of cure and storage modulus curves are similar. Both are monotonically increasing and have similar relative slopes during cross-linking. Even the inflection points are similar.

### Experiment 4

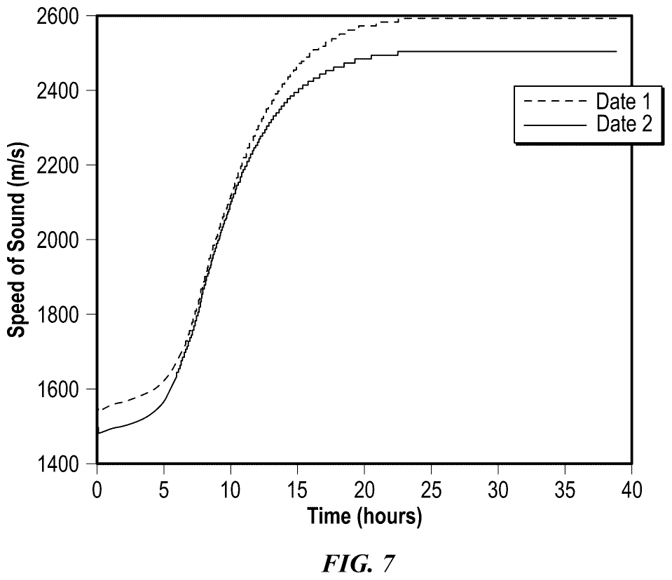

FIG. 7 is a graph of illustrative speed of sound relative to wall-clock time of two 0.5 inch resin-only samples at 22° C. There is some variability in the starting speed of sound and final speed of sound between the two test. However, the percentage difference between the maximum final speed of sound and the minimum final speed of sound is 3.5%. Much of this variability can be attributed to the tolerance in the measurement of the thickness of the sample. Interestingly, there is a continual offset between the two samples. This offset, if normalized at t=0, would be about zero. Of note, this offset should not be a fixed offset (meaning a constant vertical shift) but should be proportional to the speed of sound (i.e., the higher the speed of sound, the larger the separation between the curves). This example lends credence to the concept that the most precise degree of cure measurement will be to use the t=0 data and then normalize for a given signal. However, the data in FIG. 7 (and other figures) does not include a normalization at t=0 and therefore shows that the invention is still quite accurate even without an initial offset calibration/normalization.

### Experiment 5

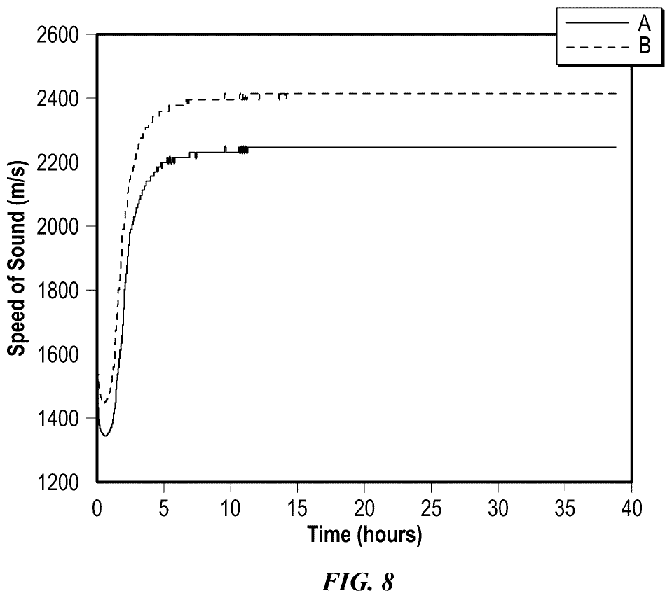

FIG. 8 is a graph of illustrative speed of sound relative to wall-clock time of two 0.25 inch resin-only samples at 40° C. There is slight variation in the initial and final speeds of sound. However, the speed of sound during portions of the test is very similar. The variation in the two final speeds of sound is 6.9%. This was the largest variation of the testing by the inventors when there is no t=0 normalization.

### Experiment 6

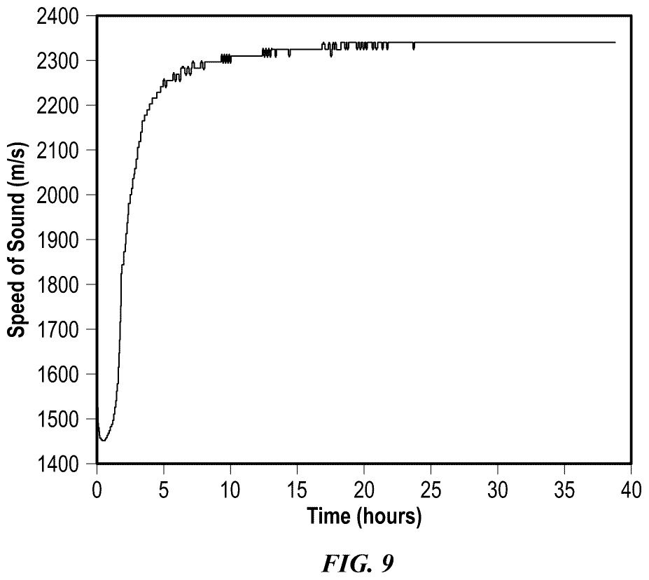

FIG. 9 is a graph of illustrative speed of sound relative to wall-clock time of one 0.25 inch carbon fiber laminate sample at 40° C. The graph is to illustrate initially different temperatures of composites. The mixed resin is initially at room temperature of about 22° C. and then is heated to 40° C. The rest of the test setup has already been heated to 40° C. in an oven at the point that the room temperature sample has been poured into the mold for monitoring. This can be seen in the dip in the first approximately 20 minutes of the data collection. Thus, for precision, such initial conditions can be incorporated into calculations when converting to the degree of cure. Such corrections can benefit from having a proper normalization if the early stages are critical. By not adjusting for such temperature changes when normalizing, empirical data suggest that an error of 5-10% can occur for the degree of cure over the range of temperatures discussed herein.

### Experiment 7

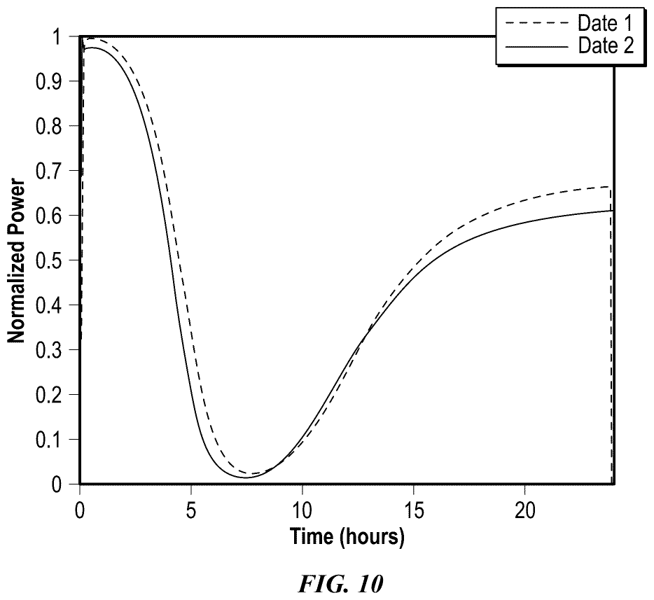

FIG. 10 is a graph of illustrative normalized power relative to wall-clock time of the ultrasound signal reflected from two 0.5 inch resin-only samples at 22° C. Throughout the tests, the normalized power is very similar to each other, except towards the ends of the tests. The inventors suggest that this variance could be due to the normalization because each test normalizes the power based on the maximum power for each individual test. The power decreases during gelation of the thermoset and increases as the sample fully cures and becomes more solid-like.

### Experiment 8

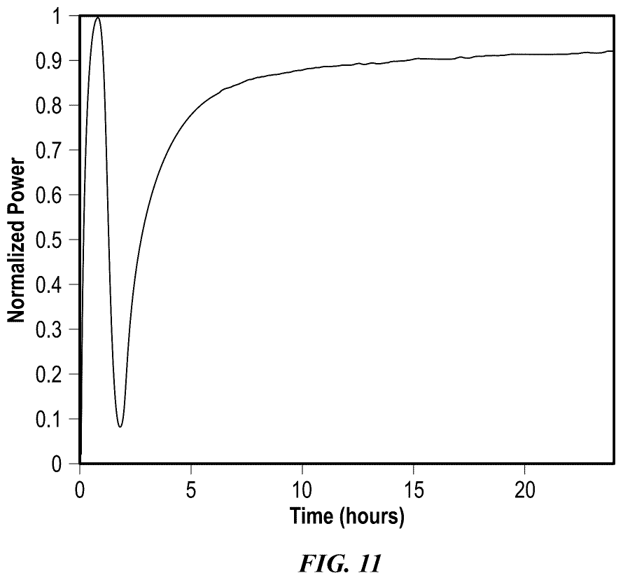

FIG. 11 is a graph of illustrative normalized power relative to wall-clock time of the ultrasound signal reflected from one 0.25 inch carbon fiber laminate sample at 40° C. Like FIG. 10, the power decreases during gelation and increases again as the material becomes more solid-like. The decrease and increase occur earlier in time than in FIG. 10 due to the increased temperature. Of note, the initial stage of pre-gelation has a high relative power, but during gelation the power of the back wall signal decreases, and then increases after gelation.

### Experiment 9

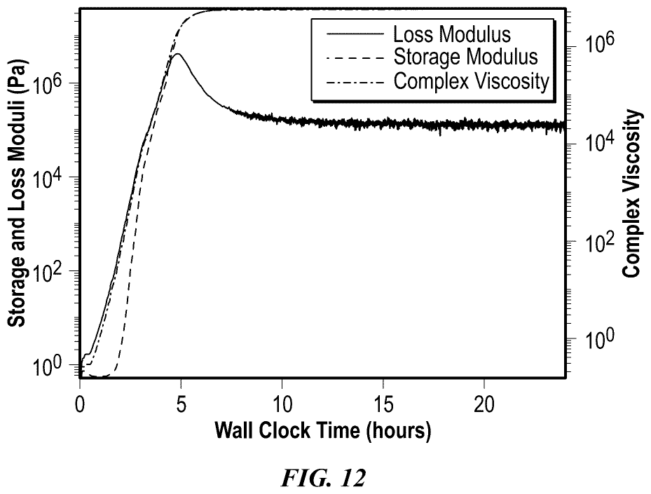

FIG. 12 is a graph of an illustrative rheological profile of parameters relative to wall-clock time of a resin-only sample at 40° C. The prior graph of rheological profile of parameters of FIG. 5 was performed at room temperature. In a similar manner, FIG. 12 illustrates the storage modulus, loss modulus, and complex viscosity over 24 hours, but at a temperature of 40° C. for comparison. The rheological plots over time is similar to FIG. 4, but the changes in storage modulus, loss modulus, and complex viscosity occur earlier in time due to the increased temperature. Specifically, the crossover point between the storage modulus and the loss modulus occurs earlier in time. These earlier in time results for the rheological parameters correspond to the ultrasound acoustic data presented in the relevant Figures with an elevated temperature, where the transition for the degree of cure occurs earlier as well.

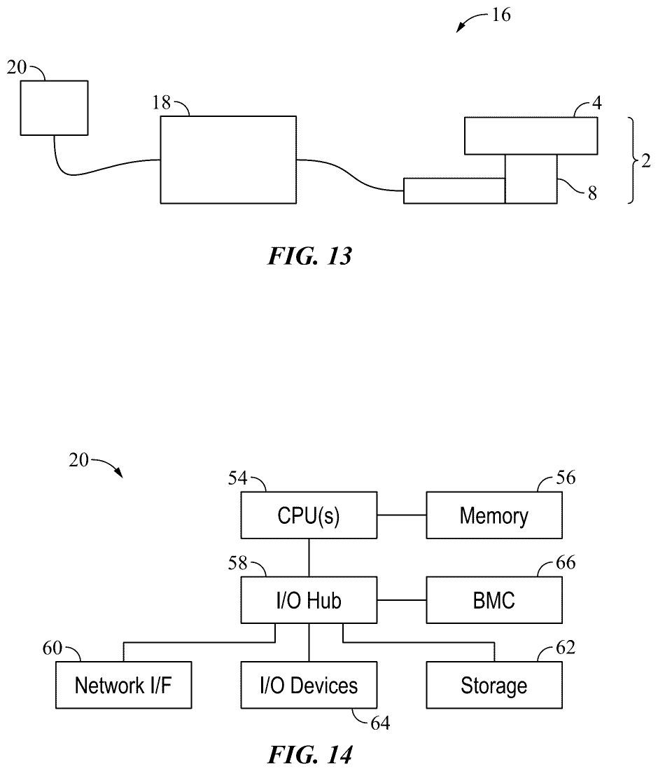

FIG. 13 is a schematic diagram of an example of an ultrasonic testing system for resin curing measurements and analysis. The ultrasonic testing system 16 incorporates the components of the ultrasonic testing setup 2 described in FIGS. 1A and 1B. Specifically, the transducer 8 can be placed adjacent to the mold 4 described in FIGS. 1A and 1B and be ultrasonically coupled with a gel to the mold. The transducer 8 can be coupled to an ultrasound data acquisition unit 18 for the transducer excitation and data capture, such as described in FIG. 1A. An analyzer 20 can be communicatively coupled with the acquisition unit to process the data, such as for the analyses described herein of the invention.

FIG. 14 is a schematic diagram of an example of the analyzer for the ultrasonic testing system of FIG. 13. The analyzer 20 can include, for example, one or more general purpose processors or central processing units (CPUs) 54 communicatively coupled to a memory resource 56 and to an input/output hub 58 to which various I/O resources and/or components are communicatively coupled. The I/O resources can include an interface 60, storage resources 62, and additional I/O devices (including components or resources) 64 including as non-limiting examples, remotely interfacing keyboards, mice, displays, printers, and so forth. The illustrated analyzer 20 can include a baseboard management controller (BMC) 66. In at least some embodiments, the BMC 66 may manage the analyzer 20 even when the analyzer is powered off or powered to a standby state. The BMC 66 may include a processor, memory, and/or other embedded information handling resources. In certain embodiments, the BMC 66 may include or may be an integral part of a remote access controller or a chassis management controller.

The system and method demonstrated in the experiments used pulse echo ultrasound to find the power in the resin-air reflection peak. The acoustic results were compared to rheological testing during the cure cycle. The acoustic power of the reflection wave was compared to the rheological testing results, and the reflection wave power minimum correlated with the onset inflection point of the loss modulus, and the inflection point of the reflection wave power inflection point during the solidification of the polymer aligned with the peak of the loss modulus. A strong correlation between the acoustic signal and the rheological testing was found by a linear correlation of the degree of cure to that of the acoustic signal speed of sound to that of the storage modulus of the resin.

Other and further embodiments utilizing one or more aspects of the inventions described above can be devised without departing from the disclosed invention as defined in the claims. For example, various resins, fibers, fillers, thicknesses, and curing profiles to produce various products, and other variations can be had than those specifically disclosed herein within the scope of the claims.

The invention has been described in the context of preferred and other embodiments and not every embodiment of the invention has been described. Obvious modifications and alterations to the described embodiments are available to those of ordinary skill in the art. The disclosed and undisclosed embodiments are not intended to limit or restrict the scope or applicability of the invention conceived of by the Applicant, but rather, in conformity with the patent laws, Applicant intends to protect fully all such modifications and improvements that come within the scope of the following claims.

What is claimed is:

1. A method for ultrasonic non-destructive testing to determine a curing state of a resin-curing product, comprising:
coupling an ultrasonic transducer to the resin-curing product;
exciting the transducer with an ultrasound data acquisition unit coupled to the transducer to produce acoustic signals in the form of waveforms and collecting data;
processing the acoustic signals into data of ultrasonic parameters; and
determining the curing state of the resin-curing product from the ultrasonic parameters.

2. The method of claim 1, wherein the processing the acoustic signals into the data of ultrasonic parameters comprises determining at least one of a speed of sound of the acoustic signals relative to a time of curing and an instantaneous peak power of the acoustic signals relative to a time of curing.

3. The method of claim 2, wherein the processing the acoustic signals into the data comprises analyzing the data for at least one of a frequency shift or a phase shift in the waveforms and adjusting the at least one of the speed of sound of the acoustic signals relative to the time of curing and the instantaneous peak power of the acoustic signals relative to the time of curing.

4. The method of claim 1, wherein collecting data further comprises:
starting data capture with an initial acoustic signal as a baseline ultrasonic parameter;
measuring further acoustic signals through the curing process to determine the ultrasonic parameters relative to the baseline ultrasonic parameter; and
using the relative ultrasonic parameters for future measurements of the resin-curing product.

5. The method of claim 1, further comprising tracking the same selected peak through the waveforms during the curing process independent of an intensity of the selected peak in the waveforms.

6. A system for ultrasonic non-destructive testing to determine a curing state of a resin-curing product, comprising:
a transducer configured in ultrasonic communication with the resin-curing product;
an ultrasound data acquisition unit coupled to the transducer and configured to excite the transducer to generate waveforms and capture data; and
an analyzer configured to analyze the waveforms into data of ultrasonic parameters indicative of the curing state of the resin-curing product.

7. A system for ultrasonic non-destructive testing to determine a curing state of a resin-curing product, comprising:
a transducer;
an ultrasound data acquisition unit coupled to the transducer and configured to excite the transducer to produce acoustic signals in the form of waveforms and collect data; and
an analyzer having a processor and a memory accessible to the processor, including processor-executable instructions that, when executed by the processor, cause the analyzer to perform operations comprising:
process the acoustic signals into data of ultrasonic parameters; and
determine the curing state of the resin-curing product from the ultrasonic parameters.
